(19) United States
(12) Patent Application Publication
(10) Pub. No.: US 2007/0038075 A1
(43) Pub. Date: Feb. 15, 2007

(54) Catheter, catheter device, and imaging diagnostic device

(75) Inventor: Michael Maschke, Lonnerstadt (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS AKTIENGESELLSCHAFT

(21) Appl. No.: 11/473,390

(22) Filed: Jun. 23, 2006

(30) Foreign Application Priority Data
Jun. 23, 2005 (DE) 10 2005 029 270.4

(51) Int. Cl. A61B 5/05

(52) U.S. Cl. 600/411

(57) ABSTRACT

The invention relates to a catheter for brachytherapy having a radiation source for generating β or γ rays. So that the catheter can be positioned as precisely as possible it is inventively proposed to provide an NMR device in the area of a free end of the catheter for generating and detecting NMR signals created through magnetic resonance of the atomic nucleus.

### CROSS REFERENCE TO RELATED APPLICATIONS

This application claims the benefits of German Patent application No. 10 2005 029 270.4 filed Jun. 23, 2005 and is incorporated by reference herein in its entirety.

### FIELD OF THE INVENTION

The invention relates to a catheter according to the claims. It further relates to a catheter device and to an imaging diagnostic device containing said catheter or catheter device.

### BACKGROUND OF THE INVENTION

A catheter of said type is known from WO 97/25102 A1. In practice the problem arises during a use of said known catheter of its frequently not being possible to position the radiation source therewith in the vessel requiring treatment with the necessary precision.

To counter this disadvantage, said known catheter has been combined with imaging devices. These can be, for example, devices with which the image is generated based on ultrasonic signals. A device of said type is known from, for example, EP 0 885 594 B1.

U.S. Pat. No. 6,377,048 B1 and U.S. Pat. No. 6,704,594 B1 describe a catheter that is provided with an NMR device for generating and detecting NMR signals created through magnetic resonance of the atomic nucleus. Said NMR device enables the production of 2- or 3-dimensional images of tissue surrounding the catheter. Undesired deposits in vessels can in particular be rendered visible thereby.

Furthermore, devices with which a catheter's position in the body can be determined are known from the prior art, for example from EP 0 776 176 B1, EP 1 034 738 B1, and EP 0 993 804 A1. Provided therein on a catheter are a plurality of position-indicating means, for example magnetic or electromagnetic transmitters or receivers, which interact with an external magnetic field. It is possible as a consequence of the interactions to draw conclusions about the position of the position-indicating means provided on the catheter within a 3-dimensional system of coordinates. It is also known from the aforementioned documents that the positional data obtained using the device can be overlaid with image data obtained from a further device. The catheter's position can therefore be exactly reproduced thereby in an image generated on the basis of the image data. The known devices do not, however, permit any therapeutic treatment, particularly of restenoses.

### SUMMARY OF THE INVENTION

The object of the invention is to eliminate the disadvantages posed by the prior art. The aim in particular is to provide a catheter, a catheter device, and an imaging diagnostic system with all of which restenoses in particular can be safely and reliably subjected to therapeutic treatment.

Said object is achieved by means of the features of the claims. Expedient embodiments of the invention will emerge from the features of the claims.

According to the invention an NMR device for generating and detecting NMR signals created through magnetic resonance of the atomic nucleus is provided in the area of the catheter's free first end.—By using the proposed catheter it is possible to render an area around it, particularly a tissue surrounding it, visible and hence to arrange a radiation source for therapeutic treatment precisely in a predefined position within the vascular system. Improved therapeutic treatment will be facilitated thereby. The time required to treat a restenosis can be reduced.

The NMR device can contain means for generating a static magnetic field. This can be a non-homogeneous magnetic field. The means for generating the static magnetic field can contain first permanent magnets, preferably two. The NMR device can furthermore contain at least one receiving coil for detecting the NMR signals and, expediently, an amplifier for amplifying the detected NMR signals. The NMR device can furthermore be rotatable around a catheter axis. That will enable the production of 3-dimensional images when there is a movement parallel to the catheter axis. NMR devices having the aforementioned features are generally known from the prior art. Reference is made to, for example, U.S. Pat. No. 6,704,594 B1 and U.S. Pat. No. 6,377,048 B1, whose disclosure content is included herein.

According to a further particularly advantageous embodiment feature a position-indicating means with which a position can be determined within a 3-dimensional system of coordinates through interactions produced when an external magnetic field is applied is provided in the area of the first end.

The external field can in particular be a magnetic field. Said magnetic field can be a magnetic or electric alternating field. The position-indicating means can, though, conceivably also be detected by means of an ultrasonic field. The position-indicating means can contain at least one but preferably three coils. Said coils can be provided with iron cores. It is possible to generate and/or receive electromagnetic signals therewith. The position-indicating means can therefore be transmitters and/or receivers of electromagnetic signals.

At least one receiving coil can also be employed as position-indicating means. That enables the receiving coil to be employed for different functionalities, thus allowing the catheter to be miniaturized.

According to a further embodiment it is provided for a magnetic field generated by at least two coils or, as the case may be, receiving coils to have a different orientation. The magnetic fields generated by the coils or, as the case may be, receiving coils expediently mutually differ by at least 30° but preferably by 60° to 90°. In particular an arrangement of the coils or, as the case may be, receiving coils mutually displaced by 60° in terms of the orientation of the magnetic fields will on the one hand enable the device to be miniaturized and, on the other hand, with the application of appropriate computing algorithms, will allow the position of the position-indicating means to be precisely determined. Reference is also made to the disclosure content of DE 695 14 238 T2, EP 1 034 738 B1, and EP 0 993 804 A1, which disclosure content is included herein.

According to a further particularly advantageous embodiment an OCT device for generating and detecting optical signals for producing optical coherence-tomographic first image data is provided in the area of the first end. An OCT device of said type is generally known from the prior art. Reference is made in this connection to WO 01/11409 A2, whose disclosure content is included herein.

According to a further advantageous embodiment it is provided for an ultrasonic device for generating and detecting ultrasonic signals for producing second image data to be provided in the area of the first end. An ultrasonic device of said type is generally known from the prior art. Reference is made in this connection to, for example, EP 0 885 594 B1 and R. J. Dickinson, “Miniature ultrasonic probe construction for minimal access surgery”, Phys. Med. Biol. 49 (2004). The disclosure content of said documents is included herein.

Specifically the combining of the inventive catheter with the ultrasonic device is especially advantageous because ultrasonic signals penetrate more deeply than optical signals into the tissue and, furthermore, because an examination will also be possible when the vascular system is being perfused with an X-ray contrast medium.

According to a further embodiment it is provided for an inflatable balloon to be provided in the area of the first end. A vessel surrounding the catheter can be blocked by means of said balloon. It can also be used to maintain the catheter at a specific position within the vascular system. It is thereby possible to inject an irrigant into the vessel through the catheter in order then to be able to register first image data by means of the OCT device and to reconstruct first images from said data.

It has furthermore proved advantageous to provide diverting means in the area of the first end. Said diverting means can contain at least one but preferably more second permanent magnets and/or electromagnets. A magnetic field generated by at least two second permanent magnets and/or electromagnets can therein have a different orientation. With the proposed diverting means the first end of the catheter can be diverted in a desired direction through the application of suitable external magnetic fields. That will make it easier to duct the first end of the catheter along a predefined path up to the vessel requiring to be therapeutically treated.

The first permanent magnets can advantageously also be employed as the diverting means. Using the first permanent magnets with a two-fold functionality will enable the catheter to be miniaturized.

The NMR device, the radiation source, where applicable the position-indicating means, the OCT device, and/or the ultrasonic device, and/or the diverting means can be provided on a long stretched-out support structure that forms the area of the first end and is connected to a flexible tube. The support structure is expediently more rigid than the tube. What is achieved thereby is that the first end of the catheter will make as frequent as possible contact with a wall of a vessel surrounding the catheter. The position and/or a path of the position-indicating means can consequently be calculated especially precisely from the data obtained from said position-indicating means.

In the context of the present invention a first or free end of the catheter is understood as being an end inserted first into the vascular system up to the vessel requiring to be therapeutically treated. The first end is expediently rounded to avoid damaging the vascular system. An area of the free or first end describes a section that contains the first end of the catheter and is necessary for accommodating in particular the position-indicating means and where applicable the OCT device, the ultrasonic device, the diverting device, and suchlike. The area of the first end contains in particular the support structure. Said area is as a rule 1 cm to 5 cm long.

According to a further embodiment it is provided for a layer surrounding the support structure and/or tube to be provided for screening magnetic fields. Said layer can contain hollow fibers or nanomagnetic particles produced from an electrically conducting material. Signal leads in particular can thereby be screened from magnetic fields generated particularly by an external source or by the coils.

According to a further embodiment it is provided for the support structure and/or tube to be provided with a marking that will be recognizable when the X-ray image is produced. That will allow the position determined using the position-indicating means to be correlated with image data assigned to a further system of coordinates for an X-ray image.

A transponder indicating the catheter's characteristics can furthermore also be provided. The will enable specific characteristics of the catheter to be remotely interrogated. Parameters for appropriately controlling the catheter can furthermore be conveyed wirelessly to an external system. Finally, information enabling the catheter to be tracked in a clinic's logistics chain can be stored in the transponder.

Provided further according to the invention is a catheter device having an internal catheter and an external catheter that is ducted within said internal catheter and to whose fifth end a radiation source is attached, and wherein in the area of the first end of the internal catheter and/or in the area of the fifth end of the external catheter an NMR device is provided for generating and detecting NMR signals created through magnetic resonance of the atomic nucleus. It can in particular be the case with the proposed alternative solution that no radiation source is provided on the internal catheter. The alternative solution first enables the internal catheter to be inserted as far as into the vessel requiring to be therapeutically treated. Said internal catheter can then be used, as it were, as a guiding means and the external catheter slid up to the vessel requiring to be therapeutically treated. The external catheter has the radiation source at the fifth end. A certain period of time is required for the vessel requiring to be therapeutically treated to be reached using the internal catheter. An exposure to radiation occurring during said period can be reduced through the provisioning of an external catheter having a radiation source attached to its fifth end.

The NMR device can contain a means for generating a static magnetic field. Said means for generating a static magnetic field contains first permanent magnets, preferably two. The NMR device can furthermore contain at least one receiving coil for detecting the NMR signals. An amplifier for amplifying the detected NMR signals can also be provided. The NMR device can furthermore be rotatable around a catheter axis. That will enable the production of 3-dimensional images. NMR devices of said type are generally known from the prior art. Reference is made to, for example, U.S. Pat. No. 6,704,594 B1 and U.S. Pat. No. 6,377,048 B1.

According to an advantageous embodiment it is provided for the radiation source to be embodied as a ring cylinder or hollow cylinder so that the internal catheter can be slid back and forth through the radiation source. That will enable subsequent insertion of the external catheter into the vessel. The advantages already mentioned can be achieved thereby.

Like the internal catheter, the external catheter can also be provided with a marking that will be recognizable when an X-ray image is produced. A layer surrounding the external catheter can furthermore be provided for screening magnetic fields. Said layer can also contain hollow fibers and/or nanomagnetic particles produced from an electrically conducting material. Reference is made in this connection to the aforementioned advantages.

The internal catheter can incidentally have the same embodiment features as the catheter. Reference is made in this respect to the explanations given above.

Provided further according to the invention is an imaging device having an inventive catheter or an inventive catheter device, with a device being provided for determining a 2- or 3-dimensional image from the NMR signals.—The proposed imaging device is suitable for diagnosing and for the ensuing therapeutic treatment particularly of restenoses. Precisely an area requiring to be therapeutically treated within the vascular system can thereby be rendered visible. Therapeutic treatment can be restricted to the desired predefined area within the vascular system, thereby minimizing patient discomfort.

According to an advantageous embodiment a means is provided for converting the NMR signals into first image data assigned to a first system of coordinates. This can be a conventional analog-to-digital converter forming part of a data-processing device. A position determined by the position-indicating means can be used for assigning the first image data to a first system of coordinates. A device for determining a feed path of the catheter or catheter device can, however, also be provided. This can be, for example, a conventional distance sensor or suchlike.

The aforementioned device for determining the position of a position-indicating means is known from the prior art. It can comprise electromagnetic transmitters or, alternatively, electromagnetic receivers that interact with the position-indicating means. Depending on the specific embodiment, the position-indicating means can be either transmitters or receivers. At least one transmitter is as a rule assigned to one receiver, or vice versa. Reference is also made in this connection to the disclosure content of the following documents that are included herein: EP 0 776 176 B1, EP 1 034 738 B1, EP 0 993 804 A1.

The device for determining expediently has at least two field generators for generating magnetic fields, in particular magnetic alternating fields of differing frequency. That will enable the position-indicating means to be localized within the 3-dimensional system of coordinates.

According to a further embodiment of the invention a device is provided for calculating a vessel center line reproducing the path of the position-indicating means. This is a 1-dimensional line in the 3-dimensional system of coordinates. It can be described by means of a polynomial equation using the coordinates that can be determined from the position of the position-indicating means. Reference is also made in this connection to the disclosure content of U.S. Pat. No. 6,546,271 B1, which content is included herein.

According to a further embodiment a means is provided for calculating a vessel envelope describing a vessel wall. That will enable, for example, a minimum and a maximum vessel diameter to be estimated and vasoconstrictions to be detected.

According to a further embodiment a device is provided for rotating the NMR device. The NMR device can be rotated thereby, preferably at a constant speed, around the catheter axis. Recordings of a vessel surrounding the catheter can thereby consequently be made circumferentially in the area of the free end of the catheter. The signals supplied by the NMR device can be evaluated in the device for rotating. The device for rotating can for that purpose include a device for evaluating the NMR signals. Evaluating herein primarily comprises digitizing the detected signals and correlating them with a specific angle of rotation.

According to a further embodiment the device for generating a 2- or 3-dimensional image contains a means for correlating the first and second system of coordinates. The first image data can thereby be referred to, for example, the second system of coordinates. That makes it possible to reduce artifacts due to divergences in the systems of coordinates. It is also possible for the first image data to be assigned directly to the second system of coordinates. Assigning to a first system of coordinates can therefore be dispensed with. The device for generating a 2- or 3-dimensional image is expediently a computer by means of which, by employing a suitable image-reconstruction program, the image data and positional data can be correlated and processed into an image that reproduces the vessel, with its being possible for image artifacts to be corrected in particular using the vessel center line determined using the position-indicating means.

According to a further embodiment of the invention a means is provided for correlating the coordinates determined by means of the position-indicating means with further coordinates. An X-ray device having at least one semiconductor detector and a data-processing device can be provided for determining the further coordinates. With the proposed embodiment it is possible to correlate, for example, images that have been generated using further imaging diagnostic devices with the images supplied using the inventive catheter or catheter device. A device can furthermore be provided for optionally overlaying a first image on the basis of the first image data and/or a second image generated by means of an imaging diagnostic device. Overlaying or fusing of said type will yield highly informative images. These can be, for example, 3-dimensional images in which the position of the catheter can be shown precisely.

The imaging diagnostic device can have been selected from the following group: X-ray device, preferably an X-ray computer tomograph; nuclear magnetic resonance tomograph; positron emission tomograph (PET); single photon emission tomograph (SPECT); endoscopic imaging device.

According to a further embodiment a device is provided for generating an external magnetic field having a predefined orientation and strength for diverting the diverting means. The imaging diagnostic device is expanded by means of the proposed device to include a further function of precisely guiding the catheter within the vascular system. The diverting means provided in the area of the free end of the catheter can for this purpose be selectively diverted by means of directed external magnetic fields and a change in the direction of the free end of the catheter to a predefined direction be achieved thereby.

It has proved expedient especially in the field of angiography to combine the proposed imaging diagnostic device with what is termed a “bi-plane X-ray device”, provided in which are at least one X-ray source, a first semiconductor detector located on a first level and a second semiconductor detector located on a second level different from the first. That makes it possible to produce large-area survey radiographs in which the catheter's position within the vascular system can be recognized particularly from the X-ray markings provided thereon.

### BRIEF DESCRIPTION OF THE DRAWINGS

Exemplary embodiments of the invention are explained in more detail below with the aid of the drawings.

FIG. 1 is a cross-sectional schematic of a catheter,

FIG. 2 is a cross-sectional schematic of the catheter shown in FIG. 1 having a drive device,

FIG. 3 is a cross-sectional schematic of a first catheter device,

FIG. 4 is a cross-sectional schematic of a second catheter device,

FIG. 5 is a cross-sectional schematic of a third catheter device,

FIG. 6 is a cross-sectional schematic of a fourth catheter device,

FIG. 7 is an overview of the main components of an imaging diagnostic device, and

FIG. 8 is a schematic of a method for generating a 3-dimensional image.

### DETAILED DESCRIPTION OF THE INVENTION

In the case of the catheter shown in FIG. 1 a free first end E1 is embodied as rounded. In the area of the first end E1 the catheter is provided with a radiation source 1 by means of which β or γ radiation can be generated for therapeutic purposes.

Position-indicating means are identified by the reference numeral 2. These can be, for example, three coils arranged in the X, Y, and Z direction mutually displaced by 90°. The coils can, however, also be arranged mutually displaced by another angle, for example 60°. Instead of the coils, other suitable transmitting or receiving means can also be provided, for example permanent magnets or ultrasonic transducers, arranged analogously mutually displaced in terms of the orientation of the magnetic flux. The reference numeral 3 identifies an inflatable balloon.

Located in a tube 4 of the catheter is a core assembly 5 that is rotatable around a catheter axis and on whose second end E2 an NMR device 6 is attached. The NMR device 6 is arranged opposite a window 7 that is permeable to magnetic fields. The NMR device 6 can be a conventional NMR device such as is known from, for example, U.S. Pat. No. 6,704,594 B1 or U.S. Pat. No. 6,377,048 B1. It can in particular have two permanent magnets for generating a static magnetic field having two different orientations, and a receiving coil. The NMR device 6 can further contain a pre-amplifier for amplifying the signals received by means of the receiving coil. A support structure T supporting in particular the radiation source 1, the position-indicating means 2, and the window 7 extends over an area containing the free end E1. The support structure T can be manufactured from, for example, a plastic material. It is expediently more rigid than the tube 4.

Supply and/or signal leads 8 connected to the NMR device 6 are integrated in the core assembly 5. Further supply and/or signal leads 9 that are connected to the position-indicating means 2 are provided in the tube 4 or on an internal wall thereof.

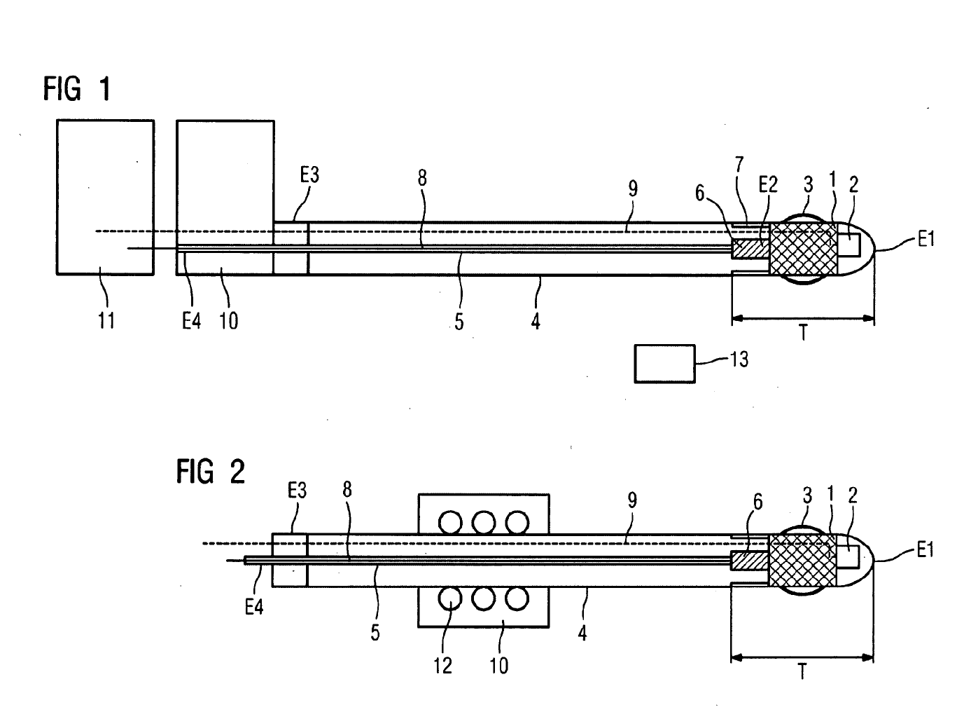

A third end E3 of the tube 4 and a fourth end E4 of the core assembly 5 are connected to a rotation device 10. As can be seen in particular from FIG. 2, the rotation device 10 can be embodied in such a way that the tube 4 is retained thereby in a frictionally engaged manner on a feeder element 12. The feeder element 12 can also be embodied in such a way that the tube 4 can be rotated thereby. The connection can be made by means of a rotation coupling enabling a supply voltage and/or signals to be coupled or, as the case may be, decoupled.

The reference numeral 11 identifies an interface by means of which the NMR signals supplied by the NMR device 6 and/or further signals supplied by the position-indicating means 2 can be digitized and assigned to a system of coordinates.

A transmitting/receiving device located outside a body requiring to be examined is identified by the reference numeral 13. A position of the position-indicating means 2 within a 3-dimensional system of coordinates can be determined computationally thereby, for example by means of a computer, and displayed, for example by means of a monitor.

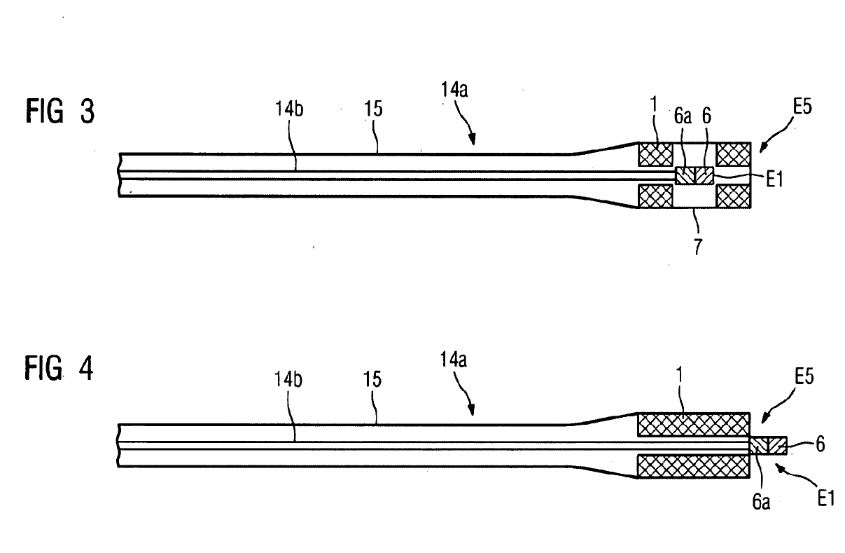

FIGS. 3 and 4 show catheter devices in the case of which a radiation source 1 is attached to a further free end E5 of the external catheter 14a. The radiation source 1 is therein embodied in the form of rings or a hollow cylinder. The external catheter 14a has a further tube 15. An internal diameter of the further tube 15 and a diameter of the rings or hollow cylinder are embodied in such a way that an internal catheter 14b of the kind shown by way of example in FIG. 4 can be ducted therethrough. The proposed catheter arrangement therefore consists of a sliding internal catheter 14b ducted within the external catheter 14a.

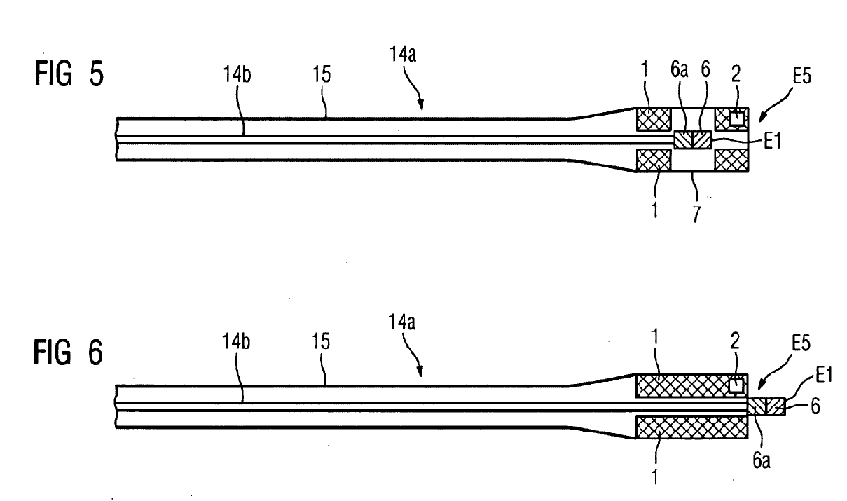

In the case of the further catheter arrangement shown in FIGS. 5 and 6 the position-indicating means 2 is attached in the area of a free fifth end E5 of the external catheter 14a. The position-indicating means 2 can in this case be omitted from the internal catheter 14b. It is, however, also possible for an internal catheter 14b according to FIG. 4 to be employed in the further catheter arrangement shown in FIG. 5 or 6. The external catheter 14a can in this case also be provided with a further position-indicating means which, compared with the position-indicating means 2, supplies distinguishable signals. As a result thereof a position of the external catheter 14a within the 3-dimensional system of coordinates can be determined separately by means of said further position-indicating means.

The further catheter arrangement shown in FIGS. 5 and 6 again features an internal catheter 14b, which is embodied, for example, according to FIG. 4, being ducted therein in a sliding manner, with a first end E1 having the NMR device 6 being able to be ducted through an opening provided on the fifth end E5 of the external catheter 14a. As can be seen from FIGS. 3 to 6, an ultrasonic device 6a is additionally provided in the area of the free end E1.

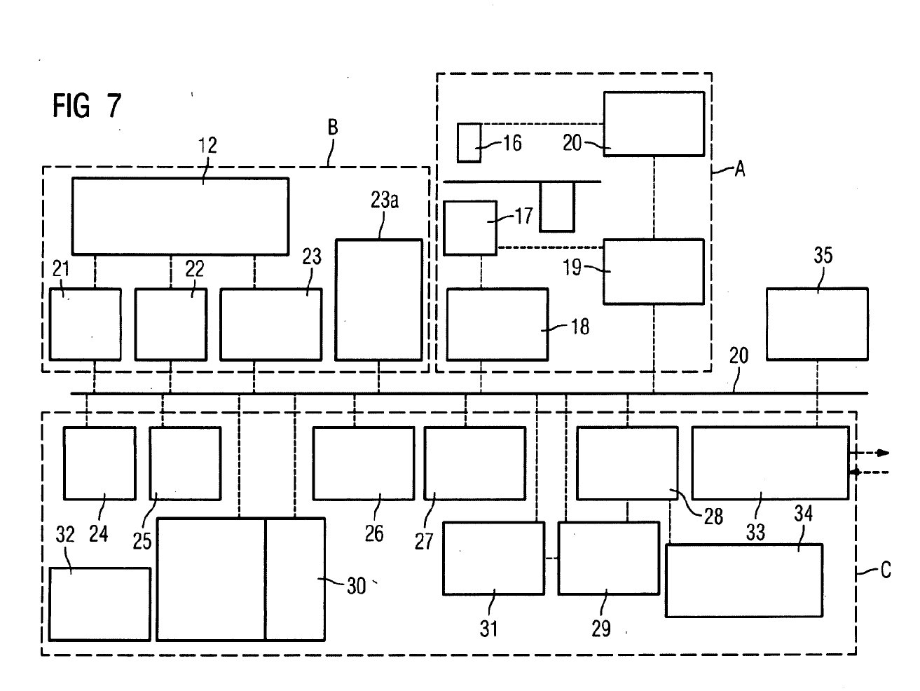

FIG. 7 is an overview of the main components of an imaging diagnostic device. The imaging diagnostic device here essentially consists of an X-ray device A, a catheter-controlling and catheter-signal-detecting device B, and a powerful data-processing device C.

The X-ray device A contains an X-ray radiating means 16, one or more X-ray detectors 17, an X-ray-image-processing unit 18, an X-ray control device 19, and a high-voltage generator 20a. The X-ray-image-processing unit 18 and the X-ray control device 19 are connected to a data bus 20.

The catheter-controlling and catheter-signal-detecting device B has the rotation device 10, 12, already described in FIG. 1, for connecting a catheter (not shown here). The rotation device 10, 12, in which digitizing of the supplied data can already be performed, is coupled to an NMR image-processing unit 21. The inventive catheter can as well as the NMR device 6 advantageously also have an ultrasonic transducer (not shown here). An ultrasonic-image-processing device 22 can be provided for evaluating the ultrasonic signals supplied by the ultrasonic transducer. A position-signal-processing device is identified by the reference numeral 23. In order to reduce motion artifacts due to, for instance, a patient's breathing or the motion of a patient's heart, sensors can be provided that detect physiological functions of said kind. A detecting unit provided for detecting and processing physiological signals supplied by the sensors is identified by the reference numeral 23a. The aforementioned units are also connected to the data bus 20.

A powerful data-processing device C enables parallel processing, in particular image processing, of the data supplied via the data bus 20. The data-processing device C can thus have, for example, a first image-processing device 24 for producing NMR images, a second image-processing device 25 for producing images from ultrasonic signals, a third image-processing device 26 for producing images from position signals, a fourth image-processing device 27 for producing X-ray images, an image-fusing and image-reconstructing unit 28, an image-correcting unit 29, and a display and control unit 30 for displaying the generated images. The image-correcting unit 29 can be connected to the data bus 20 via a calibrating unit 31. A power supply is identified by the reference numeral 32 and a further interface for importing and exporting patient data is identified by the reference numeral 33. A database in which parameter data of the X-ray radiating means or of a β, γ radiating means is stored is identified with the reference numeral 34. Finally, the reference numeral 35 identifies a data memory serving in particular to store image data.

The following typical procedural flow can be implemented using the proposed imaging diagnostic device in combination with the proposed catheter or catheter device:

Inserting the catheter or internal catheter under X-ray control, with the possibility of using a contrast medium;

Producing a radiographic, in particular an angiographic, survey;

Producing images by means of the position-indicating means;

Producing images by means of the NMR device and/or ultrasonic transducer;

Overlaying the images generated by means of the position-indicating means and using X-ray techniques;

Overlaying the images generated by means of the NMR device and/or the ultrasonic transducer with images produced using radiographic techniques;

Producing a 3-dimensional reconstruction of the images obtained by means of the NMR device and/or ultrasonic transducer using the images obtained with the position-indicating means;

Navigating the catheter or internal catheter to the target position on the basis of the generated images;

Inflating the balloon at the target position and optionally incorporating an NMR or ultrasonic contrast medium;

Generating high-resolution images by means of the NMR device and/or ultrasonic transducer in the area of the target position;

where applicable, moving an external catheter up to the target position by sliding said external catheter over the internal catheter;

Controlling the precise position of the external catheter by means of the NMR device and/or ultrasonic transducer and/or position-indicating means.

In particular the provisioning of the position-indicating means enables 3-dimensional images to be produced from the signals supplied by the NMR device and/or ultrasonic transducer. It is possible, for example, once an angiographic survey radiograph has been produced to represent the catheter's path exclusively by means of the signals supplied by the NMR device 6 and/or the ultrasonic transducer and those supplied by the position-indicating means 2 by appropriately utilizing the signals supplied by the position-indicating means, and thereby to reduce the patient's exposure to X-rays. The proposed imaging diagnostic device supplies important, in particular precise medical information about, for example, arteriosclerotic plaque and/or tumor tissue. Apart from that, the position of the free end of the catheter can be determined precisely.

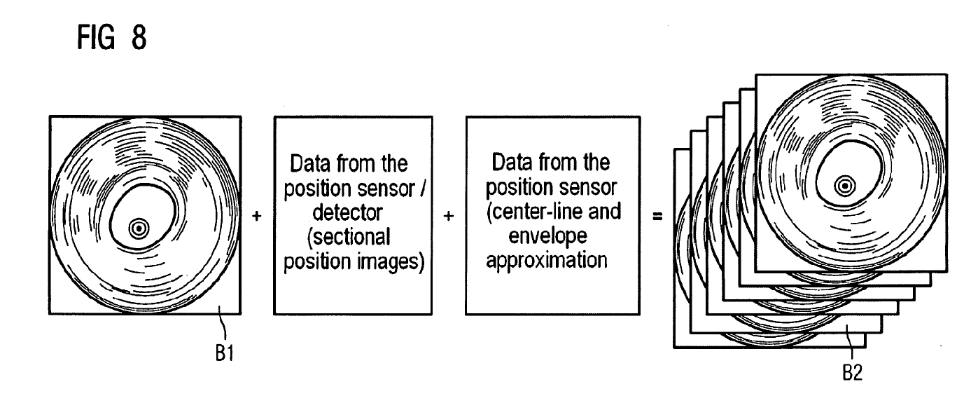

FIG. 8 is a schematic illustrating how a corrected volume data record is produced using the positional data obtained by means of the position-indicating means 2. The signals obtained by means of the NMR device 6 and/or the ultrasonic transducer can be processed into 2-dimensional first images B1. The first images B1 can also be produced by fusing images obtained from the NMR device 6 and from the ultrasonic sensor. The thus generated first images B1 can then be corrected using the positional data supplied by the position-indicating means 2. The data obtained using the position-indicating means 2 can for this purpose be computationally reconstructed using, for instance, the method of discrete tomography described in, for example, DE 102 24 011, and a 3-dimensional image calculated therefrom.

A center line of the vessel and/or an envelope thereof can furthermore be calculated from the data supplied by the position-indicating means 2. By applying said computational models the first images B1 can then be processed into a set of second images B2 having reduced artifacts compared to the first images B1.

To register or, as the case may be, overlay the patient's image data with the data obtained from the position-indicating means 2 it is necessary to transfer the spatial coordinates of the image data and of the positional data to a common system of coordinates. Any movements made by the patient while being examined can therein result in errors. To correct such errors it is possible to use a magnetic auxiliary sensor of the kind described in, for example, U.S. Pat. No. 6,233,476. An auxiliary sensor of said type can also be provided on a cable-free basis, for example by means of a Bluetooth transmitting unit. Any movements made by the patient can alternatively also be detected by means of an optical camera and determined using computational methods associated with pattern recognition, then taken into account when the image data is registered.

Separate, generally known functional units can be provided additionally in order to reduce motion artifacts due to, for instance, a patient's breathing or the motion of a patient's heart.

1-51. (canceled)

52. A catheter for brachytherapy having a first end, comprising:
a radiation source arranged in the first end of the catheter for generating β or γ rays; and
an NMR device arranged in the first end for generating and detecting NMR signals created through magnetic resonance of an atomic nucleus of a cell of a brachytherapy patient.

53. The catheter according to claim 52, wherein the NMR device comprises:
a static magnetic field generating device that contains two first permanent magnets,
a receiving coil for detecting the NMR signals, and
an amplifier for amplifying the detected NMR signals, wherein the NMR device is rotatable about a catheter axis.

54. The catheter according to claim 53, further comprising a position-indicating device arranged in the first end that determines a three-dimensional position of the catheter when an external magnetic field is applied.

55. The catheter according to claim 54, wherein the position-indicating device contains a plurality of coils having different orientations.

56. The catheter according to claim 52, wherein an inflatable balloon is arranged in the first end.

57. The catheter according to claim 56, wherein a diverting device is arranged in the first end having a plurality of second permanent magnets or second electromagnets that have a different magnetic field orientation

58. The catheter according to claim 53, wherein the first permanent magnets are a used as a diverting device that assists in guiding the catheter through the patient.

59. The catheter according to claim 58, wherein the first end comprises:
the NMR device,
the radiation source,
the position-indicating device, and
the diverting device is provided on an elongated support structure that is connected to a flexible tube.

60. The catheter according to claim 59, wherein the support structure is more rigid than the tube, and the support structure or tube is provided with a marking that is recognizable when an X-ray image is produced, and further comprising:
a layer surrounding the support structure and tube that contains hollow fibers or nano-magnetic particles produced from an electrically conducting material and screens magnetic fields, and
a transponder that indicates characteristics of the catheter.

61. A catheter device, comprising:
an external catheter having an end;
an internal catheter having an end arranged within the external catheter;
a radiation source arranged towards the end of the external catheter; and
an NMR device arranged towards the end of the internal catheter or the end of the external catheter that generates and detects NMR signals created through magnetic resonance of an atomic nucleus of a cell of a medical patient.

62. The catheter device according to claim 61 wherein the NMR device is rotatable about a catheter axis, and contains:
a static magnetic field generating device that contains two first permanent magnets,
a receiving coil for detecting the NMR signals, and
an amplifier for amplifying the detected NMR signals.

63. The catheter device according to claim 61, wherein:
the radiation source is a ring or hollow cylinder to permit the internal catheter to pass through the radiation source, and
the external catheter is provided with a marking that is recognizable when an X-ray image is produced, and
a layer surrounding the external catheter that contains hollow fibers or nano-magnetic particles produced from an electrically conducting material and screens magnetic fields.

64. The catheter device according claim 62, wherein a position-indicating device:
is arranged on the first end of the internal catheter or the external catheter,
contains three coils that have different orientations and are the receiving coils of the NMR device.

65. An imaging device, comprising:
a catheter device, comprising:
an external catheter having an end;
an internal catheter having an end arranged within the external catheter;
a radiation source arranged toward the end of the external catheter; and
an NMR device arranged at the end of the internal catheter or the end of the external catheter that generates and detects NMR signals created through magnetic resonance of an atomic nucleus of a medical patient; and
a device that generates a two-dimensional or three-dimensional image from the NMR signals.

66. An imaging device, comprising:
a catheter for brachytherapy having a free first end, comprising:
a radiation source arranged in the free end of the catheter for generating β or γ rays;
an NMR device arranged in the free end for generating and detecting NMR signals created through magnetic resonance of an atomic nucleus; and
a device that generates a two-dimensional or three-dimensional image from the NMR signals.

67. The imaging device according to claim 65, wherein the NMR signals are converted into a first image data set that is assigned to a first coordinate system.

68. The imaging device according to claim 65, wherein a position indicating device having at least two field generators for generating magnetic alternating fields of differing frequency determines a position relative to a second three-dimensional coordinate system.

69. The imaging device according to claim 66, wherein:
a centerline calculating device is provided for calculating a center line of a blood vessel by reproducing the path of the position-indicating device,
a vessel envelope calculating device is provided for calculating a blood vessel envelope that describes a blood vessel wall, and
an NMR rotating device is provided for rotating the NMR device.

70. The imaging device according to claim 66, wherein the device for generating a two-dimensional or three-dimensional image further correlates the first and second coordinate system to produce further coordinates and further comprises an X-ray device having at least one semiconductor detector and a data-processing device.

71. The imaging device according to claim 66, further comprising an overlaying device that overlays a first image with a second image generated by an imaging diagnostic device.

72. The imaging device according to claim 66, wherein the imaging diagnostic device is selected from the group consisting of:
X-ray device, preferably an X-ray computer tomograph or C- arc X-ray device;
Nuclear magnetic resonance tomograph;
Positron emission tomograph;
Single photon emission tomograph; and
Endoscopic imaging device.

73. The imaging device according to claim 66, further comprising:
a diverting device that generates an external magnetic field having a predefined orientation and strength,
an X-ray source,
a first semiconductor detector arranged on a first level, and
a second semiconductor detector arranged on a second level.
